(19) United States
(12) Patent Application Publication
(10) Pub. No.: US 2024/0277296 A1
(43) Pub. Date: Aug. 22, 2024

(54) ELECTRONIC DEVICE FOR PREDICTING MYOPIA REGRSSION AND THEREOF METHOD

(71) Applicant: VISUWORKS Inc., Seoul (KR)

(72) Inventors: Ik Hee RYU, Seoul (KR); Jin Kuk KIM, Seoul (KR); Tae Keun YOO, Seoul (KR)

(21) Appl. No.: 18/130,368

(22) Filed: Apr. 3, 2023

(30) Foreign Application Priority Data
Feb. 21, 2023 (KR) 10-2023-0023137

(51) Int. Cl. A61B 5/00; A61B 3/00; A61B 3/103; A61B 3/12; A61B 3/14

(52) U.S. Cl. CPC A61B 5/7275; A61B 3/0025; A61B 3/103; A61B 3/12; A61B 3/14

(57) ABSTRACT

An electronic device for predicting myopia regression, which includes a memory and a processor connected with the memory to execute instructions included in the memory. The processor collects first target data of a subject and second target data of the subject, extracts a first result value as output data for a first machine learning model by using the first target data as input data for the first machine learning model, and determines whether there is a possibility of myopia regression of the subject based on the first result value.

### CROSS-REFERENCE TO RELATED APPLICATION(S)

This application is based on and claims priority under 35 U.S.C. § 119 to Korean Patent Application No. 10-2023-0023137, filed on Feb. 21, 2023, in the Korean Intellectual Property Office, the disclosure of which is incorporated by reference herein its entirety.

### BACKGROUND

### 1. Field

The disclosure relates to an electronic device for predicting myopia regression and an operation method thereof.

### 2. Description of Related Art

These days, because vision correction surgery, such as laser epithelial keratomileusis/photorefractive keratectomy (LASEK/PRK), laser in situ keratomileusis (LASIK) or small incision lenticule extraction (SMILE), has an excellent effect, it is widely used to correct myopia for people with poor eyesight, regardless of gender or age. With the generalization of various IT devices such as smartphones and tablet personal computers (PCs), the number of people undergoing vision correction surgery to correct deteriorated eyesight is increasing every year. However, because the long-term effectiveness and side effects of surgery are not well known, many people are concerned about long-term complications.

The most common long-term complication after vision correction surgery is myopia regression. Because the myopia regression is generally very slow in progress speed, it is difficult to diagnose complications without long-term observation. The exact cause of the myopia regression after vision correction surgery is not known. It has been considered unpredictable because a situation is different for each person. The myopic regression entails considerable human, social, and economic costs, such as refitting glasses after vision correction surgery, using contact lenses, or performing additional vision correction surgery.

When it is able to identify a patient with a high possibility of myopia regression after vision correction surgery, human, social, and economic costs associated with complications may be reduced. Thus, there is a need for a technology capable of determining a possibility of myopia regression after vision correction surgery.

### SUMMARY

Aspects of the disclosure are to address at least the above-mentioned problems and/or disadvantages and to provide at least the advantages described below. Accordingly, an aspect of the disclosure is to provide an electronic device for predicting a possibility of myopia regression after vision correction surgery using an artificial intelligence machine learning model constructed based on preoperative data and fundus photography of a patient and an operation method thereof.

In accordance with an aspect of the disclosure, an electronic device for predicting myopia regression may include a memory and a processor connected with the memory and configured to execute instructions included in the memory. The processor may collect first target data of a subject and second target data of the subject, may extract a first result value as output data for a first machine learning model by using the first target data as input data for the first machine learning model, may extract second result value as output data for a second machine learning model by using the first result value and the second target data as input data for the second machine learning model, and may determine whether there is a possibility of myopia regression of the subject based on the second result value.

According to an embodiment of the disclosure, the first target data may be information about fundus photography of the subject. The second target data may include information about an age of the subject, a gender of the subject, a refractive power before vision correction surgery of the subject, a vision correction surgery type of the subject, intraocular pressure (IOP) before the vision correction surgery of the subject, a central corneal thickness (CCT) before the vision correction surgery of the subject, an anterior chamber depth (ACD) before the vision correction surgery of the subject, or an expected amount of cut in the vision correction surgery of the subject.

According to an embodiment of the disclosure, the processor may collect first training data, may process a first training dataset based on the first training data, may construct the first machine learning model based on the first training dataset, and may determine performance of the first machine learning model at a predetermined period. The first training data may include information about fundus photography for a plurality of subjects.

According to an embodiment of the disclosure, the processor may collect second training data, may process a second training dataset based on the second training data, may construct a second machine learning model based on the second training dataset, and may determine performance of the second machine learning model at a predetermined period. The second target data may include information about an age of each of a plurality of subjects, a gender of each of the plurality of subjects, a refractive power before vision correction surgery of each of the plurality of subjects, a vision correction surgery type of each of the plurality of subjects, intraocular pressure (IOP) before the vision correction surgery of each of the plurality of subjects, a central corneal thickness (CCT) before the vision correction surgery of each of the plurality of subjects, an anterior chamber depth (ACD) before the vision correction surgery of each of the plurality of subjects, or an expected amount of cut in the vision correction surgery of each of the plurality of subjects.

According to an embodiment of the disclosure, the processor may provide an external electronic device with whether there is the possibility of myopia regression of the subject.

In accordance with another aspect of the disclosure, an operation method of an electronic device for predicting myopia regression may include collecting first target data of a subject and second target data of the subject, extracting a first result value as output data for a first machine learning model by using the first target data as input data for the first machine learning model, extracting a second result value as output data for a second machine learning model by using the first result value and the second target data as input data for the second machine learning model, and determining whether there is a possibility of myopia regression of the subject based on the second result value.

According to an embodiment of the disclosure, the first target data may be information about fundus photography of the subject. The second target data may include information about an age of the subject, a gender of the subject, a refractive power before vision correction surgery of the subject, a vision correction surgery type of the subject, intraocular pressure (IOP) before the vision correction surgery of the subject, a central corneal thickness (CCT) before the vision correction surgery of the subject, an anterior chamber depth (ACD) before the vision correction surgery of the subject, or an expected amount of cut in the vision correction surgery of the subject.

According to an embodiment of the disclosure, the operation method may further include collecting first training data, processing a first training dataset based on the first training data, constructing a first machine learning model based on the first training dataset, and determining performance of the first machine learning model at a predetermined period. The first training data may include information about fundus photography for a plurality of subjects.

According to an embodiment of the disclosure, the operation method may further include collecting second training data, processing a second training dataset based on the second training data, constructing a second machine learning model based on the second training dataset, and determining performance of the second machine learning model at a predetermined period. The second target data may include information about an age of each of a plurality of subjects, a gender of each of the plurality of subjects, a refractive power before vision correction surgery of each of the plurality of subjects, a vision correction surgery type of each of the plurality of subjects, intraocular pressure (IOP) before the vision correction surgery of each of the plurality of subjects, a central corneal thickness (CCT) before the vision correction surgery of each of the plurality of subjects, an anterior chamber depth (ACD) before the vision correction surgery of each of the plurality of subjects, or an expected amount of cut in the vision correction surgery of each of the plurality of subjects.

According to an embodiment of the disclosure, the operation method may further include providing an external electronic device with whether there is the possibility of myopia regression of the subject.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a drawing about a myopia regression prediction system according to an embodiment of the disclosure;

FIG. 2 is a block diagram illustrating a configuration of an electronic device according to an embodiment of the disclosure;

FIG. 3A is a conceptual diagram illustrating an operation of an electronic device according to an embodiment of the disclosure;

FIG. 3B is a flowchart illustrating an operation of an electronic device according to an embodiment of the disclosure;

FIG. 3C is a conceptual diagram illustrating an operation of an electronic device according to an embodiment of the disclosure;

FIG. 4A is a flowchart illustrating an operation of an electronic device according to an embodiment of the disclosure;

FIG. 4B is a drawing illustrating an operation of an electronic device according to an embodiment of the disclosure;

FIG. 5 is a flowchart illustrating an operation of an electronic device according to an embodiment of the disclosure; and

FIG. 6 is a block diagram illustrating a hardware configuration of an electronic device for predicting myopia regression according to an embodiment of the disclosure.

### DETAILED DESCRIPTION

Hereinafter, various embodiments of the disclosure will be described with reference to accompanying drawings. However, it should be understood that the disclosure is not intended to be limited to a specific embodiment and includes various modifications, equivalents, and/or alternatives of embodiments of the disclosure. With regard to description of drawings, similar denotations may be used for similar components.

In the disclosure, the expressions “have”, “may have”, “include” and “comprise”, or “may include” and “may comprise” used herein indicate existence of corresponding features (e.g., components such as numeric values, functions, operations, or parts) but do not exclude presence of additional features.

In the disclosure, the expressions “A or B”, “at least one of A or/and B”, or “one or more of A or/and B”, and the like may include any and all combinations of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

The terms, such as “first”, “second”, “1st”, “2nd”, or the like used in the disclosure may be used to refer to various components regardless of the order and/or the priority and to distinguish the relevant components from other components, but do not limit the components. For example, without departing the scope of the disclosure, a first component may be referred to as a second component, and similarly, a second component may be referred to as a first component.

According to the situation, the expression “configured to” used in the disclosure may be used exchangeably with, for example, the expression “suitable for”, “having the capacity to”, “designed to”, “adapted to”, “made to”, or “capable of”. The term “configured to” must not mean only “specifically designed to”.

In the disclosure, for example, a “command”, an “instruction”, “control information”, a “message”, “information”, “data”, a “packet”, a “data packet”, “intent”, and/or a “signal”, transmitted and received between first electronic device(s) and second electronic device(s), may include the scope or a detailed electrical expression (e.g., a digital code/an analog physical quantity) capable of being recognized by humans irrespective of the expression or may refer to itself. It may be obvious to those skilled in the art to which the disclosure pertains that the listed exemplary expressions may be interpreted in various manners according to the used context. “A is greater than B” in the disclosure may include the meaning “A is greater than or equal to B” as well as the meaning “A is greater than B.”

Terms used in the disclosure are used to describe specified embodiments and are not intended to limit the scope of another embodiment. The terms of a singular form may include plural forms unless the context clearly indicates otherwise. All the terms used herein, which include technical or scientific terms, may have the same meaning that is generally understood by a person skilled in the art described in the disclosure. Terms, which are defined in a general dictionary, among terms used in the disclosure may be interpreted as the same or similar meaning to the meaning on context in the related art and are not interpreted as an idealized or overly formal meaning unless expressly so defined in the disclosure. In some cases, even if terms are terms which are defined in the disclosure, they may not be interpreted to exclude embodiments of the disclosure.

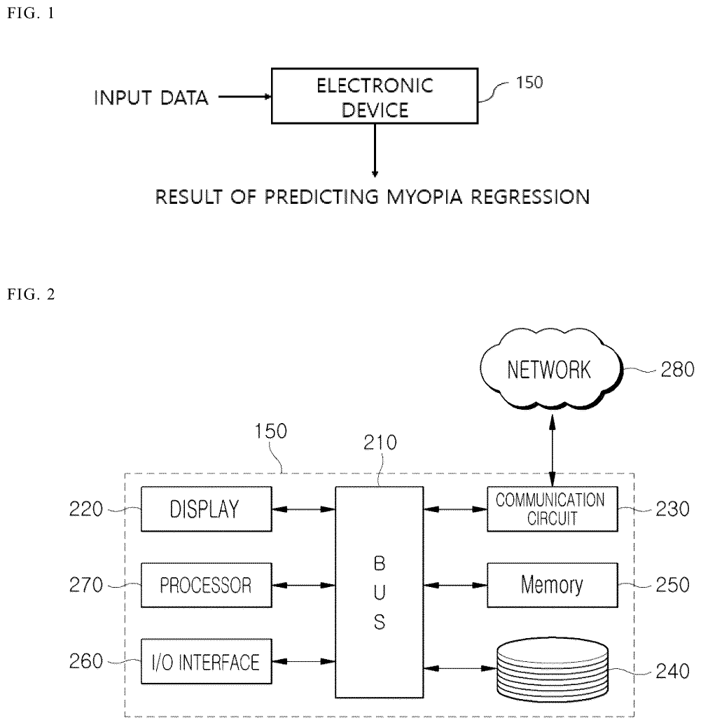

FIG. 1 is a drawing about a myopia regression prediction system according to an embodiment of the disclosure.

Referring to FIG. 1, an electronic device 150 according to an embodiment of the disclosure may communicate with another external electronic device over a network.

For reference, the electronic device 150 may be implemented as a computer capable of accessing a server or terminal in a remote place over the network. For example, the computer may include a notebook, a desktop, a laptop, or the like loaded with a web browser. Furthermore, the electronic device 150 may be implemented as a terminal capable of accessing a server or terminal in a remote place over the network. For example, the electronic device 150 may be a wireless communication device ensuring portability and mobility, which may include all types of handheld wireless communication devices, such as navigation, a personal communication system (PCS), a global system for mobile communication (GSM), a personal digital cellular (PDC), a personal handyphone system (PHS), a personal digital assistant (PDA), an international mobile telecommunication (IMT)-2000 terminal, a code division multiple access (CDMA)-2000 terminal, a wideband-code division multiple access (W-CDMA) terminal, a wireless broadband Internet (WiBro) terminal, a smartphone, a smartpad, and a tablet personal computer (PC).

The electronic device 150 according to an embodiment of the disclosure may be implemented as a computer device or a plurality of computer devices, which provide(s) a command, a code file, content, a service, or the like. The electronic device 150 may be implemented in the form of an internal webpage or application (app) created and operated by an individual or a company.

According to an embodiment of the disclosure, the electronic device 150 may receive input data from the outside and may predict myopia regression. According to an embodiment of the disclosure, the input data may include an age of a patient, a gender of the patient, a refractive power (e.g., a myopia degree, an astigmatism value, or the like) before vision correction surgery of the patient, a vision correction surgery type (e.g., laser epithelial keratomileusis/photorefractive keratectomy (LASEK/PRK), laser in situ keratomileusis (LASIK), small incision lenticule extraction (SMILE), or the like) of the patient, intraocular pressure (IOP) before the vision correction surgery of the patient, a central corneal thickness (CCT) before the vision correction surgery of the patient, an anterior chamber depth (ACD) before the vision correction surgery of the patient, an expected amount of cut in the vision correction surgery of the patient, fundus photography, and the like.

According to an embodiment of the disclosure, the electronic device 150 may identify a possibility of myopia regression based on the input data.

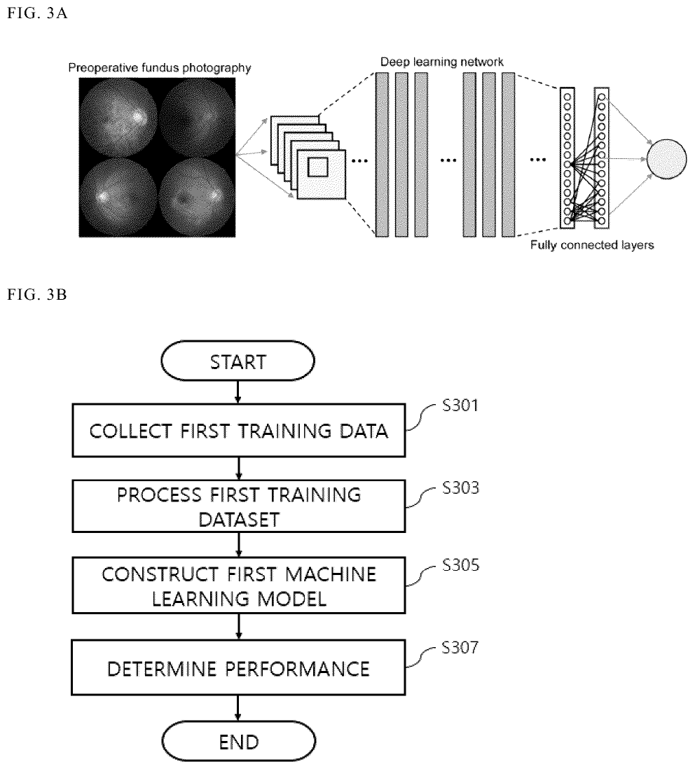

FIG. 2 is a block diagram illustrating a configuration of an electronic device according to an embodiment of the disclosure.

As shown in FIG. 2, an electronic device 150 may include a bus 210, a display 220, a communication circuit 230, a database 240, a memory 250, an input/output (I/O) interface 260, and a processor 270. In another embodiment, the electronic device 150 may exclude at least one of the components or may additionally include another component.

For reference, the components 210, 220, 230, 240, 250, 260, and 270 of the electronic device 150, which are shown in FIG. 2, are only exemplary components for describing an operation method of the electronic device 150 according to an embodiment of the disclosure. In other words, it is obvious that the electronic device 150 according to an embodiment of the disclosure is able to additionally include another component other than the shown components.

The bus 210 may electrically connect the components 220 to 270. The bus 210 may include a circuit for communication (e.g., a control message and/or data) between the components 220 to 270.

The display 220 may display text, an image, a video, an icon, a symbol, or the like configuring various pieces of content. The display 220 may include a touch screen and may receive a touch, a gesture, proximity, or a hovering input using an electronic pen or a part of the user's body.

For example, the display 220 may include a liquid crystal display (LCD), a light emitting diode (LED) display, an organic LED display, a microelectromechanical systems (MEMS) display, or an electronic paper display. The display 220 may be implemented to be included in the electronic device 150 or may be implemented independently of the electronic device 150, which may be operatively connected with the electronic device 150.

The communication circuit 230 may establish a communication channel between the electronic device 150 and external devices. The communication circuit 230 may access a network 280 through wireless communication or wired communication to communicate with the external devices.

The database 240 may be implemented on the memory 250 or may be implemented on a separate storage medium. The database 240 may store all of contents, details, or the like of data transmitted and received with the external device. The data stored in the database 240 may be updated constantly at a predetermined period.

According to an embodiment of the disclosure, the database 240 may store various pieces of information input from the external device. For example, the database 240 may store an age of a patient, a gender of the patient, a refractive power (e.g., a myopia degree, an astigmatism value, or the like) before vision correction surgery of the patient, a vision correction surgery type (e.g., such as laser epithelial keratomileusis/photorefractive keratectomy (LASEK/PRK), laser in situ keratomileusis (LASIK), small incision lenticule extraction (SMILE), or the like) of the patient, intraocular pressure (IOP) before the vision correction surgery of the patient, a central corneal thickness (CCT) before the vision correction surgery of the patient, an anterior chamber depth (ACD) before the vision correction surgery of the patient, an expected amount of cut in the vision correction surgery of the patient, fundus photography, and the like.

According to various embodiments, because the data stored in the database 240 is information sensitive to a subject, it may be distributed and stored in a blockchain network to improve security about use of the pieces of information. When the database 240 is distributed and stored in the blockchain network, a history of transmitting, modifying, deleting, or adding information included in the database 240 may be more securely managed in the blockchain network.

The memory 250 may include a volatile and/or non-volatile memory. The memory 250 may store a command or data associated with at least one other component of the electronic device 150. For example, the memory 250 may store instructions, when executed, causing the processor 270 to perform various operations described in the specification. As an example, the instructions may be included in a package file of an application program.

The I/O interface 260 may serve to deliver a command or data, input from a user or another external device, to another component of the electronic device 150. The I/O interface 260 may be implemented with hardware or software and may be used as the concept including a user interface (UI) and a port for communication with another external device.

The processor 270 may include at least one or more of a central processing unit (CPU), an application processor (AP), or a communication processor (CP). The processor 270 may be electrically connected with the memory 250, the display 220, and the communication circuit 230 through the bus 210 and may execute calculation or data processing about control and/or communication of other components depending on instructions, a program, or software stored in the memory 250, during its operation. Thus, the execution of the instructions, the application program, or the software may be understood as an operation of the processor 270.

According to an embodiment of the disclosure, the processor 270 may perform data processing or the like for performing an operation of learning and predicting a model associated with predicting myopia regression which will be described below. According to an embodiment of the disclosure, the processor 270 may learn the model associated with predicting myopia regression which will be described below and may generate the result of predicting the myopia regression using the model associated with predicting the myopia regression.

The network 280 may include at least one of a telecommunications network, a computer network, the Internet, or a telephone network. A wireless communication protocol for accessing the network 280 may use at least one of, for example, long-term evolution (LTE), LTE-advanced (LTE-A), code division multiple access (CDMA), wideband CDMA (WCDMA), universal mobile telecommunications system (UMTS), wireless broadband (WiBro), global system for mobile communications (GSM), or a 5th generation (5G) standard communication protocol. However, this is illustrative, and various wired and wireless communication technologies applicable in the technical field may be used according to an embodiment to which the disclosure is applied.

As such, the electronic device 150 according to an embodiment of the disclosure may predict myopia regression after vision correction surgery while minimizing an examination to observe the myopia regression to reduce costs and time and may identify a high-risk patient with the myopia regression to provide a subject with a customized treatment strategy.

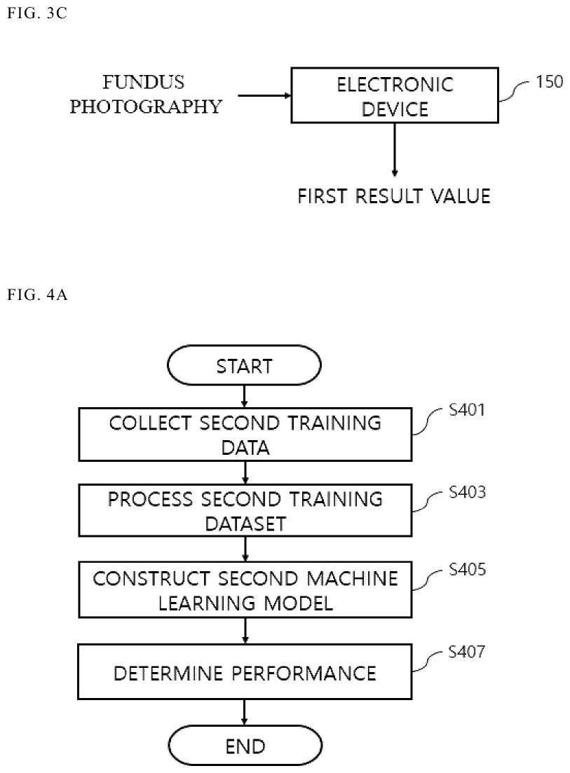

FIG. 3A is a conceptual diagram illustrating a first machine learning model constructed by an electronic device 150 according to an embodiment of the disclosure.

The first machine learning model according to an embodiment of the disclosure may include a neural network for analyzing a photo in which a fundus of the subject is captured (hereinafter referred to as “fundus photography”). According to an embodiment of the disclosure, the neural network may be composed of a set of node units connected with each other. A plurality of nodes refer to a plurality of neurons. According to an embodiment of the disclosure, the nodes constituting the neural network may be connected by one or more links. In the neural network, one or more nodes connected through the link may form a relationship between an input node and an output node.

In the relationship between the input node and the output node connected through one link, a value of data of the output node may be determined according to data input to the input node. According to an embodiment of the disclosure, the link which connects the input node with the output node may have a weight. The weight may be variable. Furthermore, the neural network according to an embodiment of the disclosure may vary a weight by a user or a certain algorithm to predict myopia regression. For example, when one or more input nodes are connected with one output node through the link, the output node may determine an output node value based on values input to input nodes connected with the output node and a weight set in the link corresponding to the respective input nodes.

According to an embodiment of the disclosure, the performance of the neural network may be determined according to the number of nodes and links in the neural network, a correlation between the nodes and the links, or a value of the weight assigned to each of the links. For example, when the number of nodes and the number of links are identical to each other and when a weight value of the link is present in different neural networks, two neural networks may be recognized as being different from each other.

The neural network may be composed of a set of a plurality of nodes, and a subset of nodes constituting the neural network may be referred to as a layer. Some of the nodes constituting the neural network may constitute a layer, based on a distance formed from a specific input node. For example, a set of a plurality of nodes with a distance of “n” from the specific input node may constitute an “n”-layer. The distance from the specific input node refers to the number of links which should pass to be reached from the specific input node to the node. However, the definition of such a layer is only an embodiment, but not limited thereto.

According to an embodiment of the disclosure, in the neural network, the number of nodes included in the input layer may be the same as the number of nodes included in the output layer. In the neural network according to another embodiment of the disclosure, the number of nodes included in the input layer may be different from the number of nodes included in the output layer.

A deep neural network (DNN) refers to a neural network including at least one or more hidden layers as well as an input layer and an output layer. Latent structures of data may be identified using the DNN. For example, latent structures of fundus photography (e.g., whether there is a specific pattern, what the retinal nerve looks like, whether the pigment layer is thin, or the like) may be identified using the DNN. The DNN may include a convolutional neural network, a recurrent neural network, a generative adversarial network, an auto encoder, a deep belief network (DBN), or the like. However, this is illustrative, but not limited thereto.

The neural network may be learned in at least one of supervised learning, unsupervised learning, semi supervised learning, or reinforcement learning. The learning of the neural network may be a process of applying information (or data) for the neural network to predict myopia regression to the neural network and constructing a specific model.

The neural network may be learned based on training data such that an output error is minimized. The neural network may repeatedly receive training data and may calculate an error between the output of the neural network and target data based on the training data. To reduce the calculated error, the neural network may perform backpropagation of the error of the neural network in a direction from an output layer (or an output node) of the neural network to an input layer (or an input node) of the neural network and may update a weight of each node of the neural network.

For supervised learning, the neural network may use training data (or labeling data) where each training data is labeled with a correct answer value. For unsupervised learning, in the neural network, each training data may fail to be labeled with a correct answer. For example, the training data used for supervised learning about data classification may be data where each of the training data is labeled with a category. The neural network may receive labeling data and may compare the output of the neural network with the label of the training data to calculate an error. For another example, the neural network may calculate an error while comparing training data which is an input used for unsupervised learning about data classification with the output of the neural network. The calculated error may be propagated in a reverse direction in the neural network. The neural network may update a weight corresponding to the link of the respective nodes included in each layer of the neural network depending on backpropagation. The amount of change in the updated connection weight of each node may be determined according to a learning rate. The calculation of the neural network for the input data and the backpropagation of the error may constitute a learning cycle. The learning rate may be applied differently according to the number of times the learning cycle of the neural network is repeated.

The training data may be generally a subset of actual data (i.e., fundus photography to be processed using the learned neural network or the like) in the learning of the neural network. Although the error for the training data is reduced, there is the case where an error increases in the actual data. Overfitting is a phenomenon where it is overlearned from the training data and an error increases in actual data. Overfitting acts as a cause of increasing errors in a machine learning algorithm.

An electronic device 150 of FIG. 2 according to an embodiment of the disclosure may use a method such as regularization for increasing training data to prevent overfitting, dropout for deactivating some of nodes of the network in the learning process, or batch normalization layer.

FIG. 3B is a flowchart illustrating an operation of an electronic device 150 according to an embodiment of the disclosure. Particularly, in FIG. 3B, an operation of the electronic device 150 for constructing a first machine learning model is illustrated according to an embodiment of the disclosure.

In operation S301, the electronic device 150 may collect first training data. According to an embodiment of the disclosure, the electronic device 150 may receive the first training data from the outside. According to another embodiment of the disclosure, the electronic device 150 may internally collect data for training. The first training data for constructing the first machine learning model may be fundus photography corresponding to a plurality of subjects.

In operation S303, the electronic device 150 may process a first training dataset based on the first training data. According to an embodiment of the disclosure, the electronic device 150 may classify the first training data into data with a high possibility of myopia regression (or abnormal data) and data with a low possibility of myopia regression (or normal data) to generate the first training dataset.

In operation S305, the electronic device 150 may construct the first machine learning model for predicting myopia regression using fundus photography of a subject based on the first training dataset.

In operation S307, the electronic device 150 may determine performance of the first machine learning model. According to an embodiment of the disclosure, the electronic device 150 may compare data output using the first machine learning model with the first training data to calculate an error. The electronic device 150 may periodically calculate the error and may determine the performance of the first machine learning model.

Although not illustrated in the drawing, the electronic device 150 may update the first machine learning model in the direction of reducing the calculated error.

FIG. 3C is a conceptual diagram illustrating an operation of an electronic device 150 according to an embodiment of the disclosure.

According to an embodiment of the disclosure, the electronic device 150 may extract a value (or a first result value) where myopia regression is predicted using a first machine learning model constructed for fundus photography (or input data).

According to an embodiment of the disclosure, the electronic device 150 may determine myopia regression of a subject by using only the first result value.

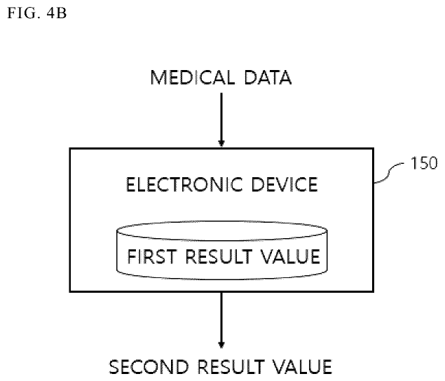

FIG. 4A is a flowchart illustrating an operation of an electronic device 150 according to an embodiment of the disclosure. Particularly, in FIG. 4A, an operation of the electronic device 150 for constructing a second machine learning model is illustrated according to an embodiment of the disclosure. Although not illustrated in the drawing, the second machine learning model may also be applied to the contents disclosed in FIG. 3A without change.

In operation S401, the electronic device 150 may collect second training data. According to an embodiment of the disclosure, the electronic device 150 may receive the second training data from the outside. According to another embodiment of the disclosure, the electronic device 150 may internally collect the second training data. The second training data for constructing the second machine learning model may include information (hereinafter referred to as “medical data”) about an age of each of a plurality of subjects, a gender of each of the plurality of subjects, a refractive power (e.g., a myopia degree, an astigmatism value, or the like) before vision correction surgery of each of the plurality of subject, a vision correction surgery type (e.g., such as epithelial laser keratomileusis/photorefractive keratectomy (LASEK/PRK), laser in situ keratomileusis (LASIK), small incision lenticule extraction (SMILE), or the like) of each of the plurality of subject, intraocular pressure (IOP) before the vision correction surgery of each of the plurality of subject, a central corneal thickness (CCT) before the vision correction surgery of each of the plurality of subject, an anterior chamber depth (ACD) before the vision correction surgery of each of the plurality of subject, or an expected amount of cut in the vision correction surgery of each of the plurality of subject and a first result value which is an output of a first machine learning model.

In operation S403, the electronic device 150 may process a second training dataset based on the second training data. According to an embodiment of the disclosure, the electronic device 150 may classify the second training data into data with a high possibility of myopia regression (or abnormal data) and data with a low possibility of myopia regression (or normal data) to generate the second training dataset. For example, the electronic device 150 may reflect the medical data in the first result value to classify the medical data into data with a high possibility of myopia regression (or abnormal data) and data with a low possibility of myopia regression (or normal data) to generate the second training dataset.

In operation S405, the electronic device 150 may construct the second machine learning model for predicting myopia regression using fundus photography of a subject and medical data of the subject based on the second training dataset. Particularly, the electronic device 150 may integrate the medical data and the first result value output using the first machine learning model and may construct the second machine learning model to predict myopia regression.

In operation S407, the electronic device 150 may determine performance of the second machine learning model. According to an embodiment of the disclosure, the electronic device 150 may compare data output using the second machine learning model with the second training data to calculate an error. The electronic device 150 may periodically calculate the error and may determine the performance of the second machine learning model.

Although not illustrated in the drawing, the electronic device 150 may update the second machine learning model in the direction of reducing the calculated error.

FIG. 4B is a conceptual diagram illustrating an operation of an electronic device 150 according to an embodiment of the disclosure.

According and an embodiment of the disclosure, the electronic device 150 may extract a value (or a second result value) where myopia regression of a subject is predicted using a second machine learning model constructed based on medical data (or input data) and a first result value.

According to an embodiment of the disclosure, the electronic device 150 may predict whether myopia regression of the subject occurs based on the second result value.

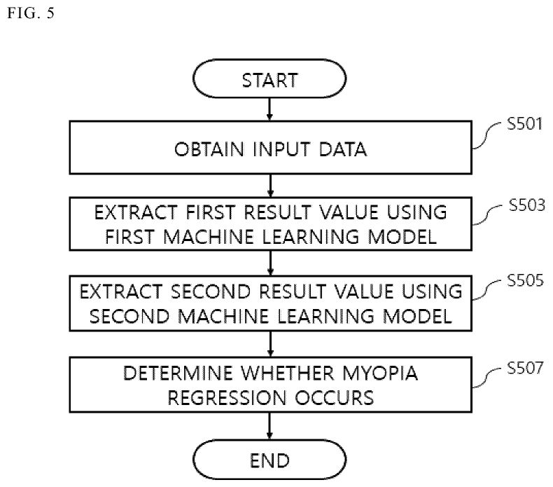

FIG. 5 is a flowchart illustrating an operation of an electronic device 150 according to an embodiment of the disclosure.

In operation S501, the electronic device 150 may collect input data from the outside. The input data may include medical data and fundus photography. According various embodiment of the disclosure, the electronic device 150 may internally collect medical data and fundus photography.

In operation S503, the electronic device 150 may extract a first result value for fundus photography of a subject using a first machine learning model.

In operation S505, the electronic device 150 may extract a second result value for the first result value corresponding to the subject and medical data for the subject using a second machine learning model.

In operation S507, the electronic device 150 may determine whether myopia regression of the subject occurs based on the extracted second result value.

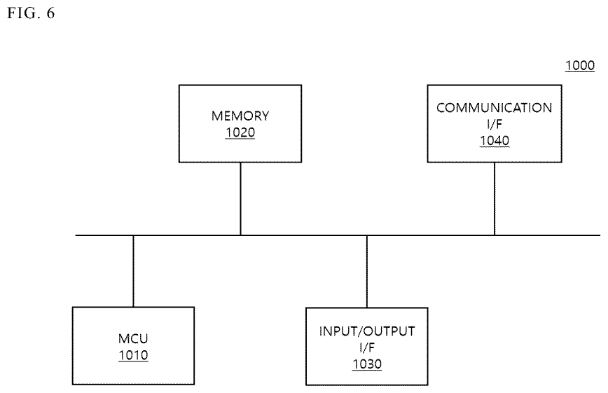

FIG. 6 is a block diagram illustrating a hardware configuration of an electronic device for predicting myopia regression according to an embodiment of the disclosure.

A computing system 1000 according to an embodiment disclosed in the disclosure may include a microcontroller unit (MCU) 1010, a memory 1020, an input/output interface (I/F) 1030, and a communication I/F 1040.

The MCU 1010 may be a processor to execute various programs for predicting myopia regression, which are stored in the memory 1020, process several pieces of data by means of such programs, and perform functions of an electronic device 150 shown in FIG. 2, which are described above.

The memory 1020 may store various programs. Furthermore, the memory 1020 may store various pieces of data received from a client.

Such a memory 1020 may be plural in number if necessary. The memory 1020 may be a volatile memory and may be a non-volatile memory. A random access memory (RAM), a dynamic RAM (DRAM), a static RAM (SRAM), or the like may be used as the memory 1020 as the volatile memory. A read-only memory (ROM), a programmable ROM (PROM), an electrically alterable ROM (EAROM), an erasable programmable ROM (EPROM), an electrically erasable programmable ROM (EEPROM), a flash memory, or the like may be used as the memory 1020 as the non-volatile memory. Examples of the listed memories 1020 are only illustrative, but not limited thereto.

The input/output I/F 1030 may provide an interface to connect between an input device (not shown), such as a keyboard, a mouse, or a touch panel, and an output device, such as a display (not shown), and the MCU 1010 and transmit and receive data.

The communication I/F 1040 may be a component capable of transmitting and receiving various pieces of data with a server, which may be various devices capable of supporting wired or wireless communication. For example, a program for managing various pieces of data, the various pieces of data, or the like may be transmitted and received with an external server, which is separately provided, through the communication I/F 1040.

As such, a computer program according to an embodiment disclosed in the disclosure may be recorded in the memory 1020 and may be processed by the MCU 1010, thus being implemented as a module which performs, for example, respective functions shown in FIG. 2.

Hereinabove, although it has been described that all components configuring the embodiment of the disclosure are combined with each other as one component or are combined and operated with each other as one component, the disclosure is not necessarily limited to the above-mentioned embodiment. In other words, all the components may also be selectively combined and operated with each other as one or more components without departing from the scope of the disclosure.

Meanwhile, various embodiments disclosed in the specification may be implemented with hardware, middleware, microcode, software, and/or a combination thereof. For example, various embodiments may be implemented in one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, other electronic units designed to perform functions presented herein, or a combination thereof.

Further, for example, various embodiments may be recorded or encoded in a computer-readable medium including commands. The commands recorded or encoded in the computer-readable medium may allow the programmable processor or another processor to perform a method, for example, when the commands are executed. The computer-readable medium includes a computer storage medium and may be any available medium which may be accessed by a computer. For example, such a computer-readable medium may include a RAM, a ROM, an EEPROM, a CD-ROM, or other optical disk storage media, a magnetic disk storage medium, or other magnetic storage devices.

The hardware, software, firmware, and the like may be implemented in the same device or individual devices so as to support various operations and functions disclosed in the specification. Additionally, in the disclosure, constituent elements, units, modules, components, and the like disclosed as “˜unit” may be individually implemented as logic devices which are operated together or individually, but may be mutually operated. Description of different features of the modules, the units, and the like is intended to emphasize different functional embodiments and does not necessarily mean that the embodiments need to be implemented by individual hardware or software components. On the contrary, functions associated with one or more modules or units may be performed by individual hardware or software components or may be integrated in common or individual hardware or software components.

Operations are illustrated in drawings in a specific order, but it should not be appreciated that the operations need to be performed in a specific order or a sequential order which is illustrated or all illustrated operations need to be performed in order to achieve a desired result. In any environment, multi-tasking or parallel tasking may be advantageous. Moreover, in the aforementioned embodiments, it should not be appreciated that various components need to be distinguished in all embodiments and it should be appreciated that the disclosed constituent elements may be generally together integrated in a single software product or packaged to a plurality of software products.

The electronic device, the server, or the external device according to various embodiments of the disclosure described above may include at least one of, for example, smartphones, tablet PCs, mobile phones, video phones, desktop PCs, laptop PCs, personal digital assistants (PDAs), portable multimedia players (PMPs), MP3 players, mobile medical devices, cameras, or wearable devices.

According to various embodiments, the wearable device may include at least one of an accessory type (e.g., watches, rings, bracelets, anklets, necklaces, glasses, contact lens, or head-mounted-devices (HMDs)), a fabric or garment-integrated type (e.g., an electronic apparel), a body-attached type (e.g., a skin pad or tattoos), or a bio-implantable type (e.g., an implantable circuit).

According to some embodiments, the electronic device or the external device may be a home appliance. The home appliances may include at least one of, for example, televisions (TVs), digital video disk (DVD) players, audios, refrigerators, air conditioners, cleaners, ovens, microwave ovens, washing machines, air cleaners, set-top boxes, home automation control panels, security control panels, TV boxes, game consoles, electronic dictionaries, electronic keys, camcorders, electronic picture frames, or the like.

According to another embodiment, the electronic device, the external device, or the wearable device may include at least one of various medical devices (e.g., various portable medical measurement devices (e.g., a blood glucose monitoring device, a heartbeat measuring device, a blood pressure measuring device, a body temperature measuring device, or the like), a magnetic resonance angiography (MRA), a magnetic resonance imaging (MRI), a computed tomography (CT), scanners, and ultrasonic devices), navigation devices, a global navigation satellite system (GNSS), event data recorders (EDRs), flight data recorders (FDRs), vehicle infotainment devices, home robots, or Internet of things (e.g., light bulbs, various sensors, electric or gas meters, sprinkler devices, fire alarms, thermostats, street lamps, exercise equipment, hot water tanks, heaters, boilers, or the like).

As described above, the best embodiment is disclosed in the drawings and specification. The terminology used herein is for the purpose of describing the disclosure only and is not intended to limit the meaning or limit the scope of the disclosure described in the claims. Therefore, those skilled in the art will understand that various modifications and other equivalent embodiments are possible from it. Consequently, the true technical protective scope of the disclosure should be determined based on the technical spirit of the appended claims.

The electronic device for predicting the myopia regression and the operation method thereof according to the disclosure may predict myopia regression after vision correction surgery while minimizing an examination to observe the myopia regression to reduce costs and time and may identify a high-risk patient with the myopia regression to provide a customized treatment strategy.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device for predicting myopia regression, the electronic device comprising:
a memory; and
a processor connected with the memory and configured to execute instructions included in the memory,
wherein the processor collects first target data of a subject and second target data of the subject, extracts a first result value as output data for a first machine learning model by using the first target data as input data for the first machine learning model, and determines whether there is a possibility of myopia regression of the subject based on the first result value.

2. The electronic device of claim 1, wherein the processor extracts second result value as output data for a second machine learning model by using the first result value and the second target data as input data for the second machine learning model and determines whether there is the possibility of myopia regression of the subject based on the second result value.

3. The electronic device of claim 1, wherein the first target data is information about fundus photography of the subject, and
wherein the second target data includes information about an age of the subject, a gender of the subject, a refractive power before vision correction surgery of the subject, a vision correction surgery type of the subject, intraocular pressure (IOP) before the vision correction surgery of the subject, a central corneal thickness (CCT) before the vision correction surgery of the subject, an anterior chamber depth (ACD) before the vision correction surgery of the subject, or an expected amount of cut in the vision correction surgery of the subject.

4. The electronic device of claim 1, wherein the processor collects first training data, processes a first training dataset based on the first training data, constructs the first machine learning model based on the first training dataset, and determines performance of the first machine learning model at a predetermined period, and
wherein the first training data includes information about fundus photography for a plurality of subjects.

5. The electronic device of claim 1, wherein the processor collects second training data, processes a second training dataset based on the second training data, constructs a second machine learning model based on the second training dataset, and determines performance of the second machine learning model at a predetermined period, and
wherein the second target data includes information about an age of each of a plurality of subjects, a gender of each of the plurality of subjects, a refractive power before vision correction surgery of each of the plurality of subjects, a vision correction surgery type of each of the plurality of subjects, intraocular pressure (IOP) before the vision correction surgery of each of the plurality of subjects, a central corneal thickness (CCT) before the vision correction surgery of each of the plurality of subjects, an anterior chamber depth (ACD) before the vision correction surgery of each of the plurality of subjects, or an expected amount of cut in the vision correction surgery of each of the plurality of subjects.

6. An operation method of an electronic device for predicting myopia regression, the operation method comprising:
collecting first target data of a subject and second target data of the subject;
extracting a first result value as output data for a first machine learning model by using the first target data as input data for the first machine learning model; and
determines whether there is a possibility of myopia regression of the subject based on the first result value.

7. The operation method of claim 6, further comprising:
extracting a second result value as output data for a second machine learning model by using the first result value and the second target data as input data for the second machine learning model; and
determining whether there is the possibility of myopia regression of the subject based on the second result value.

8. The operation method of claim 6, wherein the first target data is information about fundus photography of the subject, and
wherein the second target data includes information about an age of the subject, a gender of the subject, a refractive power before vision correction surgery of the subject, a vision correction surgery type of the subject, intraocular pressure (IOP) before the vision correction surgery of the subject, a central corneal thickness (CCT) before the vision correction surgery of the subject, an anterior chamber depth (ACD) before the vision correction surgery of the subject, or an expected amount of cut in the vision correction surgery of the subject.

9. The operation method of claim 6, further comprising:
collecting first training data;
processing a first training dataset based on the first training data;
constructing the first machine learning model based on the first training dataset; and
determining performance of the first machine learning model at a predetermined period,
wherein the first training data includes information about fundus photography for a plurality of subjects.

10. The operation method of claim 6, further comprising:
collecting second training data;
processing a second training dataset based on the second training data;
constructing a second machine learning model based on the second training dataset; and
determining performance of the second machine learning model at a predetermined period,
wherein the second target data includes information about an age of each of a plurality of subjects, a gender of each of the plurality of subjects, a refractive power before vision correction surgery of each of the plurality of subjects, a vision correction surgery type of each of the plurality of subjects, intraocular pressure (IOP) before the vision correction surgery of each of the plurality of subjects, a central corneal thickness (CCT) before the vision correction surgery of each of the plurality of subjects, an anterior chamber depth (ACD) before the vision correction surgery of each of the plurality of subjects, or an expected amount of cut in the vision correction surgery of each of the plurality of subjects.
